(19) United States
(12) Patent Application Publication
(10) Pub. No.: US 2009/0082483 A1
(43) Pub. Date: Mar. 26, 2009

(54) POLYGLYCEROL BASED POLYOLS AND POLYURETHANES AND METHODS FOR PRODUCING POLYOLS AND POLYURETHANES

(76) Inventors: Zoran S. Petrovic, Pittsburg, KS (US); Mihail Ionescu, Pittsburg, KS (US); Ivan Javni, Pittsburg, KS (US)

Correspondence Address: SPENCER, FANE, BRITT & BROWNE, 1000 WALNUT STREET, SUITE 1400, KANSAS CITY, MO, 64106-2140, US

(21) Appl. No.: 12/233,199

(22) Filed: Sep. 18, 2008

Related U.S. Application Data
(60) Provisional application No. 60/973,960, filed on Sep. 20, 2007.
(60) Provisional application No. 61/086,964, filed on Aug. 7, 2008.

(51) Int. Cl. C08G 18/72; C07C 69/708; C07C 29/00

(52) U.S. Cl. 521/172; 560/182; 568/852; 528/74.5

(57) ABSTRACT

A new class of polyols derived from renewable resources, including polyglycerol and vegetable oils, the use of such polyols in polyurethane foams and cast resins, and methods for making the polyols and polyurethanes are provided.

### CROSS REFERENCE TO RELATED APPLICATIONS

This Application is related to earlier-filed Provisional Applications Ser. No. 60/973,960, filed Sep. 20, 2007, and Ser. No. 61/086,964, filed Aug. 7, 2008. The identified earlier-filed applications are hereby incorporated by reference into the present Application.

### BACKGROUND OF THE INVENTION

The present invention relates to polyols derived from renewable resources and methods for making the polyols. Further, the present invention includes using polyols derived from renewable resources to produce polyurethane foams and resins.

Polyether polyols produced from raw materials derived from petroleum are reacted with di- or poly-isocyanates to produce polyurethane compositions useful in a variety of applications, including coatings, adhesives, sealants, elastomers, and flexible or rigid foams. Vegetable oil polyols obtained by various methods (e.g., hydroformylation of vegetable oils, ring opening of epoxy groups of epoxidized soybean oil, hydrogenation of epoxidized soybean oil) or castor oil, having hydroxyl numbers in the range of 30-600 mg KOH/g, can be reacted with aromatic isocyanates to provide polyurethane foams. Polyols characterized by high functionalities, for example, 3 or more hydroxyl groups per molecule, having hydroxyl numbers in the range of approximately 300-600 mg KOH/g, are particularly useful for production of rigid polyurethane foams.

Polyglycerol is a high functionality polyether polyol that can be produced by a variety of chemical processes, including for example, reaction of glycerol with epichlorohydrin, reaction of glycerol with glycidol, and by self-condensation of glycerol in the presence of acid or base catalysts, with elimination of water. Polyglycerol is a complex mixture of linear, branched and cyclic oligomers, where the relative size and distribution of the oligomers varies according to the method of making. Synthetic pathways from epichlorohydrin or glycidol generally tend to optimize the amount of linear oligomers, while acid catalyzed polycondensation of glycerol tends to optimize the amount of branched and cyclic oligomers. Due to the relatively high cost of glycerol historically, fatty acid esters of polyglycerol were used primarily as biodegradable surfactants, primarily in the food and cosmetics industry. Generally the linear polyglycerol esters are more biodegradable than the esters of highly branched or cyclic polyglycerol. Therefore, production methods were directed toward higher cost routes to obtain polyglycerol with very low content of branched or cyclic oligomers, such as the SOLVAY process (U.S. Pat. No. 5,041,688). Generally polyglycerol is insoluble in common organic media such as ethers, ketones, aromatic compounds, halogenated compounds, etc. Only high polarity solvents such as water, alcohols, and aprotic dipolar solvents (DMF or DMSO) can be used as solvents for polyglycerol.

Due to relatively high cost and limited compatibility with many organic materials, polyglycerol has not previously been widely used in the polyurethane industry. More recently, the high level of worldwide interest in bio-diesel fuel production has lead to an increase in production and availability of glycerol, which has resulted in glycerol becoming increasingly cost competitive with other simple polyols.

### BRIEF SUMMARY OF THE INVENTION

There are, therefore, provided in the practice of the invention new high functionality polyol compositions and polyurethane compositions based on polyglycerol. There are also provided in the practice of the invention improved methods for producing the polyols and polyurethanes. Polyglycerol, a polyhydroxyl compound of high functionality and of high hydroxyl number, is used to prepare improved polyols of high functionality and high hydroxyl number by esterification with aliphatic and aromatic carboxylic acids and by transesterification with lower alkyl esters of aliphatic and aromatic carboxylic acids, and vegetable oil derivatives. Such polyols are useful for application in rigid polyurethane foams. Polyglycerol produced by self-polycondensation of glycerol derived from bio-diesel production is 100% from renewable resources. As a consequence, the polyols obtained from vegetable oil derivatives and polyglycerol are 100% from renewable resources.

In an embodiment of the invention polyols suitable for use in rigid polyurethane foams are obtained by transesterification of vegetable oil polyols with polyglycerol at elevated temperatures in the presence of tin or titanium catalysts, wherein substantially all of the ester groups are equilibrated with substantially all of the hydroxyl groups in the reaction system. The transesterification of vegetable oil polyols with polyglycerol produces a mixture of polyols having a high hydroxyl number (300-450 mg KOH/g), high functionality (4-8 OH groups/mol), and viscosities of approximately 4-25 Pa.s at 25° C. In an embodiment, lower viscosity polyols are obtained by transesterification of polyglycerol with castor oil (4-5 Pa.s at 25° C. for a polyol with hydroxyl number of approximately 340 mg KOH/g).

In accordance with one embodiment of the present invention, polyols suitable for use in rigid polyurethane foams are prepared by transesterification of lower alkyl esters of unsaturated fatty acids, including the methyl esters of oleic acid, linoleic acid, and linolenic acid, mixtures of unsaturated and saturated fatty acids, including methyl soyate, or lower alkyl esters of hydroxyl-containing fatty acids, including methyl-ricinoleate and methyl-9(10)-hydroxy-10(9)-methoxy-stearate, with polyglycerol at a temperature range of approximately 160° C. to approximately 250° C. in the presence of a catalytic amount of potassium methoxide. In another embodiment, the lower alkyl fatty acid esters are transesterified with polyglycerol in the presence of a tin containing catalyst, whereby the resulting polyols, without further workup, can then be reacted with di- or poly-isocyanates to produce polyurethanes of desired properties.

In accordance with another embodiment of the invention, hyper-branched polyols are prepared by co-polycondensation of glycerol with either trimethylol propane or pentaerythritol in the presence of an alkaline catalyst, such as potassium methoxide, and transesterification with vegetable oils, including castor oil or soybean oil. The resulting polyols are then neutralized and reacted with di- or poly-isocyanates to produce polyurethanes of desired properties.

In accordance with another embodiment of the invention, high functionality polyols are produced directly by reaction of glycerol with vegetable oils, including castor oil or soybean oil, in the presence of catalysts at temperatures up to approximately 270° C., whereby the poly-condensation of glycerol and transesterification with the vegetable oil ester groups occur simultaneously. The polyols thus produced are reacted with di- or poly-isocyanates to produce the desired polyurethanes.

Accordingly, it is an object of the present invention to provide improved high functionality polyols from renewable resources, including glycerol, vegetable oils, and esters of fatty acids, high performance polyurethanes based on such polyols, and improved methods for making such polyols and polyurethanes.

The invention is capable of embodiments in addition to those described and of being practiced and carried out in various ways. Also, it is to be understood that the phraseology and terminology employed herein, as well as the abstract, are for the purpose of description and should not be regarded as limiting.

### BRIEF DESCRIPTION OF THE DRAWINGS

Further features of the present invention will become apparent to those skilled in the art to which the present invention relates from reading the following description with reference to the accompanying drawings, in which:

FIG. 1 is a schematic diagram of an example apparatus useful in the process of producing polyols of the invention.

FIG. 2 is a schematic diagram of the transesterification of polyglycerol with a vegetable oil polyol.

### DETAILED DESCRIPTION OF THE INVENTION

The polyols derived from renewable resources and the polyurethanes of the invention will now be described in detail with reference to the examples set forth below, and methods for making the polyols will be described with reference to the drawing figures, in which like reference numerals refer to like steps throughout.

Vegetable oil polyols, including castor oil, and vegetable oil based polyols obtained by various chemical methods, including or example, ozonolysis of vegetable oils, hydroformylation of vegetable oils, ring opening of epoxy groups of epoxidized vegetable oils, hydrogenation of epoxidized vegetable oils, having hydroxyl numbers in the range of approximately 60 to approximately 300, or more, mg KOH, can be reacted with aromatic di- or poly-isocyanates (such as MDI or PAPI) to provide polyurethanes. If such vegetable oil polyols are used in a mixture with glycerol in order to sufficiently increase functionality for production of rigid polyurethane foams, the processing of the foams is complicated by the insolubility of glycerol in the reaction mixture resulting in foams with inconsistent product properties.

An embodiment in accordance with the present invention provides novel polyols useful for production of superior rigid polyurethane foams. The polyols of the invention have high functionalities (4 to >8 OH groups/mol) with hydroxyl numbers in the range of approximately 300-600 mg KOH/g. In order to increase the hydroxyl numbers and the functionalities of vegetable oil polyols, while maximizing renewable resource utilization, a new class of polyols is provided by transesterification of vegetable oil based polyols with polyglycerol.

Polyglycerol is typically a complex mixture of linear, branched, and cyclic oligomers. Polyglycerol oligomers useful in the invention are prepared by poly-condensation of glycerol in the presence of alkaline catalysts at temperatures in the range of approximately 240° C. to 270° C., preferably approximately 250° C., for approximately 2 hours to 10 hours, preferably about 4 hours to 6 hours. Exemplary catalysts include the Group I metal hydroxides, preferably NaOH or KOH; the Group I metal methoxides, preferably potassium methoxide, and the Group II metal hydroxides, preferably Ca(OH)₂. Under these conditions, the lower reactivity of secondary beta-hydroxyl group leads primarily to linear oligomers with a relatively low content of branched oligomers, and approximately 10% or less of cyclic oligomers. Alternatively, useful polyglycerols can be similarly prepared using acid catalysts, including fluoroboric acid and trifluoromethane sulfonic acid. Acid catalyzed polycondensation of glycerol produces dark colored products which are useful where dark colored polyurethanes are acceptable.

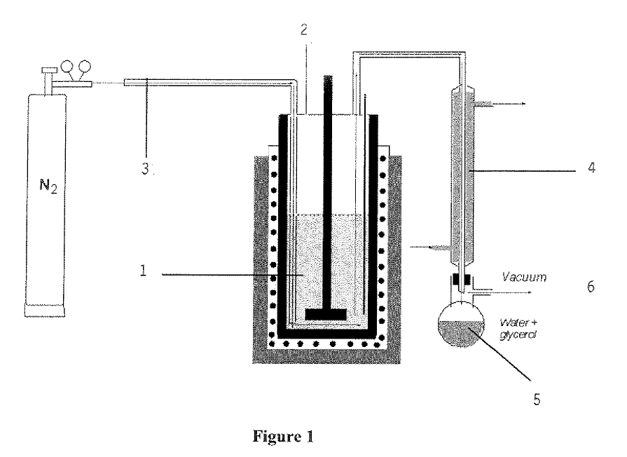

In an embodiment, the apparatus shown in FIG. 1 is charged with approximately 300 g of glycerol and approximately 0.5% to 1.2% by weight alkaline catalyst, preferably approximately 0.7% to 1% by weight alkaline catalyst. The reaction materials are charged to electrically heated reactor 1 with closable charging port 2, having means of automatic regulation of temperature (not shown). The reactor is heated to temperature with introduction of a continuous controlled flow of nitrogen 3, and condensate is swept through water cooled condenser 4, for condensing the water resulting from the reaction, into receiver 5. Optionally, vacuum source 6, may be used to further remove water of condensation, or reduce the level of unreacted glycerol. The reaction mass is heated for approximately 4-6 hours at 250° C., with continuous stirring, and continuous flow of nitrogen at approximately 250 ml/min. The distillate resulting from the polycondensation reaction is collected continuously, the volume of which increases with time. In one embodiment, the resulting crude, alkaline polyglycerol is cooled, diluted with approximately 20% to 50% by weight water, and treated with approximately 3% to 5% by weight of strong acid cation exchange resin, such as Rohm and Haas Company's AMBERLITE® 120, with stirring for about 30-45 minutes, or until the ph of the mixture is reduced to approximately 5-6. The mixture is then filtered and the water is removed by vacuum distillation at approximately 100° C. to 110° C.

Examples of the polyglycerols produced in accordance with the invention, having from 2 to approximately 20 glyceryl repeating units, are shown in Table 1.

TABLE 1
Polyglycerol by Alkaline and Acid Catalyzed Polycondensation

| | | | OH# | Visc. | | | | | |
|---|---|---|---|---|---|---|---|---|---|
| | | Time | mg | Pa · s | Polyglycerol Oligomer Distribution (%) | | | | |
| No. | Catalyst | (Hr.) | KOH/g | 25° C. | Mono | Di | Tri | Tetra | Higher |
| 1-1 | NaOH | 4 | 1052 | 12.8 | 24 | 36.3 | 20.8 | 10.4 | 7.6 |
| 1-2 | NaOH | 6 | 894 | 42.9 | 7.8 | 11.7 | 15.1 | 12.1 | 53.4 |
| 1-3 | KOH | 4 | 1117 | 13.3 | 13.8 | 31 | 23.8 | 14.2 | 17 |
| 1-4 | KOH | 6 | 910 | 52 | 10 | 18 | 15 | 12.2 | 44.8 |
| 1-5 | Ca(OH)₂ | 4 | 975 | 19.8 | 13.5 | 28.1 | 22.3 | 14 | 22 |
| 1-6 | Ca(OH)₂ | 6 | 969 | 40 | 11.9 | 20.1 | 25.7 | 17 | 25.3 |
| 1-7 | CH₃ONa | 6 | 985 | 25.5 | 10.8 | 26.4 | 21.7 | 14.6 | 26.4 |
| 1-8 | CH₃OK | 4 | 893 | 54.1 | 8 | 17.2 | 14.8 | 13.6 | 46.4 |
| 1-9 | RbOH | 6 | 988 | 17.9 | 9.9 | 8.6 | 16.7 | 26.2 | 38 |
| 1-10 | LiOH | 4 | 1036 | 3.4 | 36.4 | 52.6 | 9.5 | | 1.4 |
| 1-11 | CsOH | 6 | 991 | 5.1 | 58 | 25.4 | 10.2 | 4.3 | 2 |
| 1-12 | Sr(OH)₂ | 4 | 1018 | 4 | 7.3 | 62.2 | 21.5 | 6.5 | 2.4 |
| 1-13 | Ba(OH)₂ | 4 | 1000 | 3.6 | 36.6 | 42.8 | 14.3 | 4.6 | 1.5 |
| 1-14 | CH₃OK | 9* | 1226 | 21.3 | 16.3 | 28.8 | 22.1 | 13.7 | 18.9 |
| 1-15 | CF₃SO₃H | 4 | 1011 | 1.4 | 62.3 | 30.3 | 6.3 | | 1.1 |
| 1-16 | CH₃OK | 4 | 1098 | 18 | 1 | 39 | 26 | 16 | 18 |

| | | | | | |
|---|---|---|---|---|---|
| 1-17 | CH₃OK | 8** | 1216 | 21.4 | Average Functionality: about 4.6 |
| | | | | | hydroxyl groups per molecule |
| 1-18 | CH₃OK | 10** | 993 | 88.7 | Average Functionality: about 7.4 |
| | | | | | hydroxyl groups per molecule |

*Batch size for this example was approximately 2000 grams glycerol.
**In these examples 1660 grams glycerol and 16.6 grams solid potassium methoxide were charged in a 3 liter reaction flask. The reaction mass was heated at 250° C. under continuous flow of nitrogen of about 300 ml/min. The water of condensation was collected in a cylinder and measured as a function of time. After about 8 hours reaction time 199 ml of water were collected, and after about 10 hours reaction time 273 ml of water were collected.

Examples 1-17 and 1-18 show that the hydroxyl number and average functionality of polyglycerol correlate with the amount of water removed during condensation. By measuring the quantity of collected water as a function of time it is possible to obtain, in a controllable manner, polyglycerols with different desired hydroxyl numbers aid functionalities.

The synthesized polyglycerols can be used for esterification without removal of the alkaline catalyst.

In another embodiment, hyper-branched polyglycerol is produced by co-polycondensation of glycerol with a tri- or higher hydroxyl compound, having three or more primary hydroxyl groups, in the same manner described above. Exemplary polyhdroxy compounds for preparing hyper-branched polyglycerols include trimethylol propane or pentaerythritol. The weight ratio of glycerol to trimethylol propane or pentaerythritol is in the range of approximately 90:10 to approximately 20:80, preferably approximately 80:20 to approximately 60:40. Examples using potassium methoxide as catalyst are shown in Table 2. In one embodiment, the resulting crude, alkaline polyglycerol is cooled, diluted with approximately 20% to 50% by weight water, and treated with approximately 3% to 5% by weight of strong acid cation exchange resin, such as Rohm and Haas Company's AMBERLITE® 120, with stirring for about 30-45 minutes, or until the pH of the mixture is reduced to approximately 5-6. The mixture is then filtered and the water is removed by vacuum distillation at approximately 100° C. to 110° C.

TABLE 2
Hyper-branched Polyglycerol by Alkaline Catalyzed Polycondensation

| | | | OH# | Visc. | Polyglycerol Oligomer Distribution | | | | |
|---|---|---|---|---|---|---|---|---|---|
| | | Time | mg | Pa · s | (%) | | | | |
| No. | Comonomer | (Hr.) | KOH/g | 25° C. | Mono | Di | Tri | Tetra | Higher |
| 2-1 | Trimethylol | 4 | 1005 | 6.2 | 10.6 | 12.6 | 26.2 | 20.6 | 20 |
| | propane | | | | | | | | |
| | (20%) | | | | | | | | |
| 2-2 | Pentaerythritol | 4 | 1011 | 41.8 | 1.1 | 39.4 | 26.6 | 15.6 | 17.2 |
| | (20%) | | | | | | | | |
| 2-3 | Trimethylol | 4 | 946 | 50.6 | 3.2 | 8.7 | 30.6 | 20.9 | 36.6 |
| | propane | | | | | | | | |
| | (40%) | | | | | | | | |

In another embodiment, modified polyglycerol having lower hydroxyl number and higher viscosity is produced by catalyzed co-polycondensation of glycerol with a glycol at approximately 240° C. Useful glycols include ethylene glycol, diethylene glycol, triethylene glycol, propanediol, butanediol, hexanediol, and bisphenol A. Examples are shown in Table 2A.

TABLE 2A
Modified Polyglycerol by Acid Catalyzed Polycondensation

| | | | | OH# | Acid | |
|---|---|---|---|---|---|---|
| | | | | mg | Value mg | Visc. Pa · s |
| No. | Comonomers (grams) | | Catalyst (grams) | KOH/g | KOH/g | 25° C. |
| 2-4 | Glycerol | Diethylene | p-toluene sulfonic | 389 | 5.6 | >1000 |
| | (45) | Glycol (5) | acid (0.075) | | | |
| 2-5 | Glycerol | Diethylene | p-toluene sulfonic | 437 | 4.4 | 155 |
| | (40) | Glycol (10) | acid (0.075) | | | |

While polyglycerol can be used in a physical mixture with vegetable oil polyols for production of polyurethane foams, the insolubility of polyglycerol in the mixture often leads to inconsistent product properties, and requires extremely high intensity mixing in the foaming processes.

The foregoing problems are substantially overcome by the invention, where polyols are prepared by reaction of polyglycerol with vegetable oil derivatives prior to polyurethane foam formation. The process of transesterification of polyglycerol with vegetable oil derivatives is conducted in the presence of a suitable catalyst at approximately 160-250° C. Vegetable oil derivatives useful in the invention include: natural oils, such as canola oil, castor oil, coconut oil, corn oil, cottonseed oil, linseed oil, olive oil, palm oil, peanut oil, rapeseed oil, safflower oil, sesame oil, soybean oil, sunflower oil, etc.; chemically modified vegetable oils, including vegetable oil polyols; and lower alkyl esters of fatty acids derived from vegetable oils. Useful polyols are also produced by reaction of polyglycerol with a mixture of two or more vegetable oil derivatives. Animal fats and oils, fish oils and algae oils may be used in like manner.

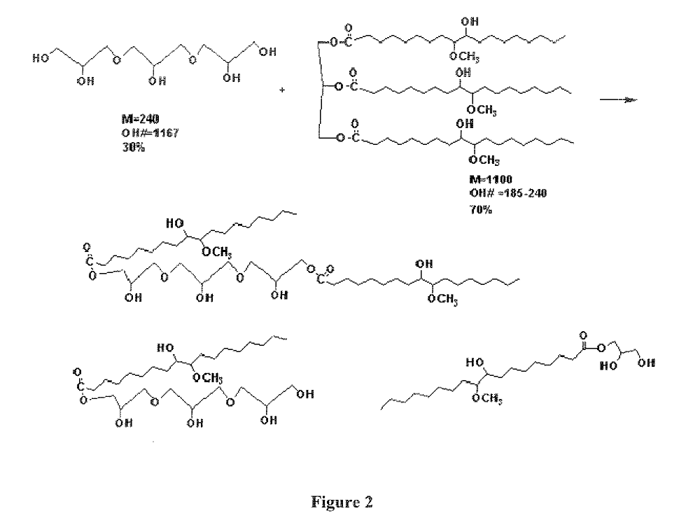

An embodiment of the invention includes polyols prepared by transesterification of approximately 40% to 95%, preferably approximately 60% to 70%, by weight vegetable oil derivatives with approximately 5% to 60%, preferably approximately 30% to 40%, by weight polyglycerol, by heating for approximately 2-10 hours, preferably about 4-6 hours, at approximately 160-250° C., preferably about 170-190° C., in the presence of approximately 0.01 to approximately 1.5 weight percent of a catalyst, including tin catalysts (e.g. Arkema FASCAT® 4350, dibutyl tin oxide, dibutyl tin dilaurate, staous octoate, etc.), titanium catalysts (e.g. titanium tetrabutoxide, titanium tetra iso-propoxide, etc.), or alkali-metal alkoxides (e.g., sodium methoxide, potassium methoxide, etc.). Substantially all of the ester groups are equilibrated with substantially all of the hydroxyl groups in the reaction system. A simplified reaction scheme for the synthesis of polyols by transesterification of glycerol trimer with glyceryl-tri-9(10)-hydroxy-10(9)-methoxy-stearate is shown in FIG. 2.

In another embodiment improved polyols of high functionality and high hydroxyl number are prepared in similar manner by esterification of polyglycerol with C-1 to C-12 aliphatic and aromatic carboxylic acids and by transesterification with lower alkyl esters of C-1 to C-12 aliphatic and aromatic carboxylic acids.

In one embodiment, approximately 0.05 to approximately 0.5 weight percent, preferably approximately 0.08 to approximately 0.12 weight percent, of an organo-tin catalyst, including FASCAT 4350, is used to catalyze the transesterification reaction. The products thus produced need no further processing prior to reaction with di- or poly-isocyanates to produce rigid polyurethane foams.

In another embodiment, alkaline polyglycerol from the alkaline catalyzed polycondensation of glycerol is used in the transesterification reaction without additional catalyst.

The resulting polyols have a high hydroxyl number (approximately 300-450 mg KOH/g), high functionality (about 4-8 OH groups/mol), and are viscous products (approximately 15-25 Pa.s at 25° C.). Lower viscosity products are obtained by transesterification with castor oil as the vegetable oil polyol (approximately 4-5 Pa.s at 25° C. for a polyol with approximately 340 mg KOH/g hydroxyl number). The resulting polyols are cloudy products, with substantially no tendency of sedimentation, while a physical mixture polyglycerol and vegetable oil polyol separates rapidly into two layers, the bottom layer being polyglycerol. The viscosity of the polyols by transesterification can be reduced substantially, and the polyols made substantially transparent, by including 5%-10% by weight low molecular weight organic additives such as dimethyl methyl phosphonate (“DMMP”) or propylene carbonate (“PC”). PC provides transparency and viscosity reduction by heating the cloudy polyol with PC for approximately 1 hour at 170° C., as a consequence of the reaction of PC with polyol hydroxyl groups in the presence of the catalysts.

In another embodiment, castor oil used in the transesterification reaction with polyglycerol leads directly to low viscosity polyols of high hydroxyl number, suitable for use in rigid polyurethane foams.

The following Table 3 describes examples of polyols derived by transesterification of vegetable oil derivatives with polyglycerols, where Polyol-173 is glyceryl-tri-9(10)-hydroxy-10(9)-methoxy-stearate having a hydroxyl number of about 160 to about 180, Polyol-204 is glyceryl-tri-9(10)-hydroxy-10(9)-methoxy-stearate having a hydroxyl number of about 185 to about 215, and Polyglycerol-3 is SOLVAY® POLYGLCEROL-3. The transesterification was conducted at approximately 170° C., except as otherwise noted in Table 3. In examples 3-1 through 3-10, approximately 0.1% by weight FASCAT 4350 was added as catalyst. In examples 3-11 through 3-22, polyglycerols from alkaline catalyzed polycondensation of glycerol were used as prepared, without neutralization, and no additional catalyst was added. In Example 3-23, approximately 1.4% by weight potassium methoxide was added as the catalyst.

TABLE 3
Polyols by Transesterification Of Polyglycerol With Vegetable Oil Derivatives

| | Vegetable | | | | Viscosity | | |
|---|---|---|---|---|---|---|---|
| | Oil | Polyglycerol | Time | | Pa · s at | Acid | |
| No. | (grams) | (grams) | (Hours) | Hydroxyl No. | 25° C. | No. | Mₙ |
| 3-1 | Polyol- | Polyglycerol-3 | 6 | 421 | 19.7 | 1.17 | 839 |
| | 173 | (60) | | | | | |
| | (140) | | | | | | |
| 3-2 | Polyol- | Polyglycerol-3 | 6 | 385 | 20.3 | 3.2 | 910 |
| | 204 | (60) | | | | | |
| | (140) | | | | | | |
| 3-3 | Castor Oil | Polyglycerol-3 | 6 | 335 | 4.1 | 1.9 | 862 |
| | (140) | (60) | | | | | |
| 3-4 | Castor Oil | Polyglycerol-3 | 6* | 340 | 4.3 | 1.8 | 890 |
| | (140) | (60) | | | | | |
| 3-5 | Methyl | Polyglycerol-3 | 5* | 365 | 7.2 | 0.5 | 599 |
| | Soyate | (100) | | | | | |
| | (150) | | | | | | |
| 3-6 | Methyl | Polyglycerol-3 | 4 | 380 | 5.2 | 0.35 | 620 |
| | soyate | (100) | | | | | |
| | (200) | | | | | | |
| 3-7 | Methyl | Polyglycerol-3 | 4 | 320 | 3.4 | 0.9 | 610 |
| | soyate | (100) | | | | | |
| | (200) | | | | | | |
| 3-8 | Methyl | Polyglycerol-3 | 4 | 273 | 3.0 | 1.1 | 660 |
| | soyate | (100) | | | | | |
| | (300) | | | | | | |
| 3-9 | 9(10)- | Polyglycerol-3 | 6 | 480 | 18.0 | 1.1 | 712 |
| | methylol- | (100) | | | | | |
| | methyl- | | | | | | |
| | stearate, | | | | | | |
| | (200) | | | | | | |
| 3-10 | 9(10) | Polyglycerol-3 | 6 | 540 | 26.8 | 1.8 | 680 |
| | methylol | (100) | | | | | |
| | methyl | | | | | | |
| | stearate | | | | | | |
| | (150) | | | | | | |
| 3-11 | Methyl | Example 1-7 | 4* | 191 | 6.2 | 0.9 | 621 |
| | Soyate | (100) | | | | | |
| | (150) | | | | | | |
| 3-12 | Methyl | Example 2-1 | 4 | 490 | 8.8 | 0.8 | 650 |
| | soyate | (100) | | | | | |
| | (100) | | | | | | |
| 3-13 | Methyl | Example 2-2 | 4 | 500 | 18.7 | 1.1 | 680 |
| | soyate | (100) | | | | | |
| | (100) | | | | | | |
| 3-14 | Methyl | Example 2-3 | 4 | 460 | 19.8 | 0.9 | 660 |
| | soyate | (100) | | | | | |
| | (100) | | | | | | |
| 3-15 | 9(10) | Example 1-4 | 6 | 412 | 15.9 | 1.0 | 780 |
| | hydroxy | (100) | | | | | |
| | 10(9) | | | | | | |
| | methoxy | | | | | | |
| | methyl | | | | | | |
| | stearate | | | | | | |
| | (200) | | | | | | |
| 3-16 | Methyl | Example 1-3 | 6 | 511 | 5.8 | 0.7 | 670 |
| | ricinoleate | (100) | | | | | |
| | (200) | | | | | | |
| 3-17 | 9(10) | Example 1-8 | 6 | 357 | 21.2 | 1.2 | 770 |
| | hydroxy | (100) | | | | | |
| | 10(9) | | | | | | |
| | methoxy | | | | | | |
| | methyl | | | | | | |
| | stearate | | | | | | |
| | (300) | | | | | | |
| 3-18 | Polyol- | Example 1-4 | 6 | 418 | 25.8 | 1.5 | 880 |
| | 173 | (100) | | | | | |
| | (200) | | | | | | |
| 3-19 | Polyol- | Example 1-4 | 6 | 439 | 28.7 | 0.9 | 930 |
| | 204 | (100) | | | | | |
| | (200) | | | | | | |
| 3-20 | Castor oil | Example 1-6 | 6 | 390 | 4.8 | 1.6 | 920 |
| | (250) | (100) | | | | | |
| 3-21 | Methyl | Example 1-16 | 4* | 213 | 3.5 | 1.08 | 618 |
| | Soyate | (100) | | | | | |
| | (150) | | | | | | |
| 3-22 | 9(10) | Example 1-3 | 4* | 1098 | 8.3 | 0.5 | 649 |
| | hydroxy | (100) | | | | | |
| | 10(9) | | | | | | |
| | methoxy | | | | | | |
| | methyl | | | | | | |
| | stearate | | | | | | |
| | (150) | | | | | | |
| 3-23 | Methyl | Polyglycerol-3 | 5* | 355 | 10.1 | 3.9 | 600 |
| | Soyate | (100) | | | | | |
| | (150) | | | | | | |

*Transesterification temperature for this example was approximately 220° C. to 230° C.

Further embodiments of the invention are described by the untested examples listed in Table 3A and Table 3B. The following examples are provided for the purpose of illustration not limitation, and the changes reflected are equally applicable to each of the compositions described in Examples 3-1 through 3-23. In examples 3A-1 through 3A-8 and 3B-1 through 3B-8, polyglycerols from alkaline catalyzed polycondensation of glycerol were used as prepared, without neutralization, and no additional catalyst was added. In examples 3A-9 through 3A-20 and 3B-9 through 3B-16, polyglycerols from alkaline catalyzed polycondensation of glycerol were first neutralized, and then approximately 0.05% to approximately 0.5% by weight FASCAT 4350 catalyst was added.

TABLE 3A
Untested Examples of Polyols by Transesterification
Of Polyglycerol With Vegetable Oil Derivatives

| | Vegetablel | Polyglycerol | | Time & |
|---|---|---|---|---|
| No. | Oi (grams) | (grams) | Catalyst | Temperature |
| 3A-1 | Castor Oil | Example 1-8 | CH₃OK | 6 hours, 160° C. |
| | (120) | (180) | | |
| 3A-2 | Castor Oil | Example 1-8 | CH₃OK | 4 hours, 190° C. |
| | (120) | (180) | | |
| 3A-3 | Castor Oil | Example 1-8 | CH₃OK | 6 hours, 160° C. |
| | (285) | (15) | | |
| 3A-4 | Castor Oil | Example 1-8 | CH₃OK | 4 hours, 190° C. |
| | (285) | (15) | | |
| 3A-5 | Polyol-204 | Example 1-4 | KOH | 6 hours, 160° C. |
| | (120) | (180) | | |
| 3A-6 | Polyol-204 | Example 1-4 | KOH | 4 hours, 190° C. |
| | (120) | (180) | | |
| 3A-7 | Polyol-204 | Example 1-4 | KOH | 6 hours, 160° C. |
| | (285) | (15) | | |
| 3A-8 | Polyol-204 | Example 1-4 | KOH | 4 hours, 190° C. |
| | (285) | (15) | | |
| 3A-9 | Castor Oil | Example 1-8 | FASCAT 4350 | 6 hours, 160° C. |
| | (120) | (180) | | |
| 3A-10 | Castor Oil | Example 1-8 | FASCAT 4350 | 4 hours, 190° C. |
| | (120) | (180) | | |
| 3A-11 | Castor Oil | Example 1-8 | FASCAT 4350 | 6 hours, 160° C. |
| | (285) | (15) | | |
| 3A-12 | Castor Oil | Example 1-8 | FASCAT 4350 | 4 hours, 190° C. |
| | (285) | (15) | | |
| 3A-13 | Polyol-173 | Example 1-5 | FASCAT 4350 | 6 hours, 160° C. |
| | (120) | (180) | | |
| 3A-14 | Polyol-173 | Example 1-5 | FASCAT 4350 | 4 hours, 190° C. |
| | (120) | (180) | | |
| 3A-15 | Polyol-173 | Example 1-5 | FASCAT 4350 | 6 hours, 160° C. |
| | (285) | (15) | | |
| 3A-16 | Polyol-173 | Example 1-5 | FASCAT 4350 | 4 hours, 190° C. |
| | (285) | (15) | | |
| 3A-17 | Soybean Oil | Example 2-1 | FASCAT 4350 | 6 hours, 160° C. |
| | (120) | (180) | | |
| 3A-18 | Soybean Oil | Example 2-1 | FASCAT 4350 | 4 hours, 190° C. |
| | (120) | (180) | | |
| 3A-19 | Soybean Oil | Example 2-1 | FASCAT 4350 | 6 hours, 160° C. |
| | (285) | (15) | | |
| 3A-20 | Soybean Oil | Example 2-1 | FASCAT 4350 | 4 hours, 190° C. |
| | (285) | (15) | | |

TABLE 3B
Untested Examples of Polyols by Transesterification
Of Polyglycerol With Carboxylic Acid Esters

| | Carboxylic Acid Ester | Polyglycerol | | Time & |
|---|---|---|---|---|
| No. | (grams) | (grams) | Catalyst | Temperature |
| 3B-1 | Methyl acetate | Example 1-8 | CH₃OK | 6 hours, 160° C. |
| | (120) | (180) | | |
| 3B-2 | Methyl acetate | Example 1-8 | CH₃OK | 4 hours, 190° C. |
| | (120) | (180) | | |
| 3B-3 | Methyl acetate | Example 1-8 | CH₃OK | 6 hours, 160° C. |
| | (285) | (15) | | |
| 3B-4 | Methyl acetate | Example 1-8 | CH₃OK | 4 hours, 190° C. |
| | (285) | (15) | | |
| 3B-5 | Methyl benzoate | Example 1-4 | KOH | 6 hours, 160° C. |
| | (120) | (180) | | |
| 3B-6 | Methyl benzoate | Example 1-4 | KOH | 4 hours, 190° C. |
| | (120) | (180) | | |
| 3B-7 | Methyl benzoate | Example 1-4 | KOH | 6 hours, 160° C. |
| | (285) | (15) | | |
| 3B-8 | Methyl benzoate | Example 1-4 | KOH | 4 hours, 190° C. |
| | (285) | (15) | | |
| 3B-9 | Methyl pelargonate | Example 1-8 | FASCAT 4350 | 6 hours, 160° C. |
| | (120) | (180) | | |
| 3B-10 | Methyl pelargonate | Example 1-8 | FASCAT 4350 | 4 hours, 190° C. |
| | (120) | (180) | | |
| 3B-11 | Methyl pelargonate | Example 1-8 | FASCAT 4350 | 6 hours, 160° C. |
| | (285) | (15) | | |
| 3B-12 | Methyl pelargonate | Example 1-8 | FASCAT 4350 | 4 hours, 190° C. |
| | (285) | (15) | | |
| 3B-13 | Methyl acetate | Example 1-5 | FASCAT 4350 | 6 hours, 160° C. |
| | (120) | (180) | | |
| 3B-14 | Methyl acetate | Example 1-5 | FASCAT 4350 | 4 hours, 190° C. |
| | (120) | (180) | | |
| 3B-15 | Methyl acetate | Example 1-5 | FASCAT 4350 | 6 hours, 160° C. |
| | (285) | (15) | | |
| 3B-16 | Methyl acetate | Example 1-5 | FASCAT 4350 | 4 hours, 190° C. |
| | (285) | (15) | | |

In an embodiment, propylene carbonate (“PC”) or dimethyl methyl-phosphonate (“DMMP”) may be used as an additive to decrease the viscosity and substantially eliminate turbidity of the polyols of the invention, as shown in Table 4.

TABLE 4
Modified Polyols with Improved Clarity and Reduced Viscosity

| | | | | | Visc., | |
|---|---|---|---|---|---|---|
| | | DMMP | PC | | Pa · s, | Acid |
| No. | Polyol | wt. % | wt. % | OH No. | 25° C. | No. |
| 4-1 | Example 3-2 | — | 10 | 344 | 7.04 | 1.9 |
| 4-2 | Example 3-3 | — | 10 | 386 | 1.47 | 0.55 |
| 4-3 | Example 3-2 | 5 | — | 365 | 10.8 | 1.8 |
| 4-4 | Example 3-3 | 5 | — | 319 | 3.1 | 2 |

In an embodiment of the invention, polyols of high functionality and high hydroxyl number comprise esters of polyglycerol derived by esterification with C-1 to C-12 aliphatic and aromatic carboxylic acids or anhydrides. In another embodiment esters of polyglycerol are derived by a two step process comprising, in the first step transesterification with lower alkyl esters of C-12 to C-22 carboxylic acids, and in the second step esterification with C-1 to C-12 aliphatic and aromatic carboxylic acids or anhydrides. Examples are shown in Table 5, and properties of the polyols are shown in Table 5A.

TABLE 5
Polyols by Esterification Of Polyglycerol With
Carboxylic Acids & Anhydrides

| | | | | | Calcu- |
|---|---|---|---|---|---|
| | | | | | lated |
| | | Acid or | | | OH |
| | Polyglycerol | Anhydride | Catalyst | Reaction | Conver- |
| No. | (grams) | (grams) | (grams) | Conditions | sion, % |
| 5-1 | Example 1-17 | Acetic | None | 3 hours at | 70 |
| | (50) | anhydride | | 155° C. | |
| | | (38.9) | | | |
| 5-2 | Example 1-17 | Butyric acid | None | 14 hours at | 50 |
| | (50) | (48.02) | | 155° C. | |
| 5-3 | Example 1-17 | Hexanoic acid | p- | 10 hours at | 50 |
| | (50) | (63.5) | toluene | 180° C. | |
| | | | sulfonic | | |
| | | | acid | | |
| | | | (0.03) | | |
| 5-4 | Example 1-17 | Hexanoic acid | p- | 7 hours at | 30 |
| | (50) | (38.1) | toluene | 180° C. | |
| | | | sulfonic | | |
| | | | acid | | |
| | | | (0.03) | | |
| 5-5 | Example 1-17 | Octanoic acid | p- | 6 hours at | 30 |
| | (50) | (47.3) | toluene | 200° C. | |
| | | | sulfonic | | |
| | | | acid | | |
| | | | (0.06) | | |
| 5-6 | Example 1-17 | Methyl soyate | CH₃OK | 3 hours at | 26 |
| | (60) | (50); Acetic | (0.5) | 220° C.; 4 | |
| | | acid (17.3) | | hours at | |
| | | | | 145° C. | |
| 5-7 | Example 1-18 | Methyl soyate | CH₃OK | 4 hours at | 38 |
| | (50) | (35.1); Acetic | (0.5) | 220° C.; 5 | |
| | | anhydride | | hours at | |
| | | (22) | | 160° C. | |
| 5-8 | Example 1-18 | Methyl soyate | CH₃OK | 4 hours at | 44 |
| | (50) | (35.1); Acetic | (0.5) | 220° C.; 5 | |
| | | anhydride | | hours at | |
| | | (17) | | 160° C. | |

TABLE 5A
Properties of Polyglycerols Esterified with Carboxylic Acids & Anhydrides

| | Theoretical | Actual | Acid | Viscosity | | |
|---|---|---|---|---|---|---|
| Example | OH # | OH # | value | Pa · s | Functionality | Solubility* |
| Example | 215 | 424 | 0.9 | 1.48 | 2.3 | Cloudy in water; |
| 5-1 | | | | | | Phase separation |
| | | | | | | in toluene |
| Example | 348 | 442 | 1.6 | 0.74 | 2.7 | Soluble in both |
| 5-2 | | | | | | water and toluene |
| Example | 296 | 328 | 10.7 | 0.22 | 2.4 | Soluble in both |
| 5-3 | | | | | | water and toluene |
| Example | 522 | 539 | 15.6 | 0.77 | 3.3 | Soluble in both |
| 5-4 | | | | | | water and toluene |
| Example | 469 | 489 | 4.5 | 0.92 | 3.2 | Soluble in both |
| 5-5 | | | | | | water and toluene |
| Example | 483 | 430 | 0.5 | 2.48 | 3.4 | Cloudy in both |
| 5-6 | | | | | | water and toluene |
| Example | 342 | 312 | 4.29 | 10.2 | 4.3 | Soluble in both |
| 5-7 | | | | | | water and toluene |
| Example | 297 | 235 | 3.39 | 3.5 | 3.4 | Cloudy in water; |
| 5-8 | | | | | | Soluble in toluene |
| Example | | 1216 | | 21.4 | 4.6 | Soluble in water; |
| 1-17 | | | | | | Phase separation |
| | | | | | | in toluene |

*Polyols are rated soluble if they remain clear after adding 0.2 gram water to 2.0 grams of the polyol, or 2.0 grams toluene to 2.0 grams of the polyol.

In another embodiment of the invention, the polyols are provided in a single step by poly-condensation of approximately 5% to 60%, preferably approximately 30% to 50%, by weight glycerol, with approximately 40% to 95%, preferably approximately 50% to 70% by weight of a vegetable oil derivative, including vegetable oils or vegetable oil fatty acid methyl esters, in the presence of alkaline catalysts, under the same reaction conditions used for the synthesis of polyglycerol. In this manner two simultaneous reactions occur: polycondensation of glycerol and transesterification of hydroxyl groups with ester groups. The resulting polyols are then neutralized by adding isopropyl alcohol and a strong acid ion-exchange resin (AMBERLITE 120, H+ form), preferably with continuous stirring. The resulting slurry is then filtered to remove the ion exchange resin, and the isopropyl is removed by vacuum distillation.

In one embodiment, direct synthesis of polyglycerol fatty acid esters is accomplished by reacting about 100 g of glycerol with about 200 g of methyl soyate and approximately 0.6% to approximately 1.5% by weight of alkaline catalyst, preferably approximately 1.0% to approximately 1.3% by weight of potassium methoxide as the catalyst. Continuous flow of nitrogen and stirring were maintained at a temperature of approximately 220° C.-270° C., preferably approximately 230° C., for about 4-6 hours. Another embodiment is similarly provided, by the reaction of about 150 g of glycerol with about 150 g of Soybean oil in the presence of potassium methoxide as the catalyst at approximately 230° C.-270° C., preferably approximately 250° C., with substantially continuous flow of nitrogen and stirring, for about 4 to 6 hours. The resulting polyols are then neutralized by adding isopropyl alcohol and a strong acid ion-exchange resin (AMBERLITE 120, H+ form), preferably with continuous stirring. The resulting slurry is then filtered to remove the ion exchange resin, and the isopropyl is removed by vacuum distillation. In additional embodiments of the invention, other known vegetable oils, animal oils, or natural oil derived fatty acid methyl esters are substituted for soybean oil or methyl soyate to produce useful polyols.

Examples of polyglycerol fatty acid esters prepared by the direct method are described in the following Table 6.

TABLE 6
Polyols by Polycondensation of Glycerol With Vegetable Oil Derivatives

| | Reactants | | | | | | |
|---|---|---|---|---|---|---|---|
| | (grams), | | OH no. | Viscosity | Acid | | |
| No. | Catalyst | Time (Hr) | mg KOH/g | Pa · s., 25° C. | Value | Mₙ | M_{w} |
| 6-1 | Glycerol | 4 | 292 | 2.9 | 0.5 | 709 | 911 |
| | (100) | | | | | | |
| | Methyl | | | | | | |
| | soyate (200) | | | | | | |
| | CH₃OK | | | | | | |
| 6-2 | Glycerol | 6 | 247 | 8.6 | 1.1 | 621 | 932 |
| | (150) | | | | | | |
| | Soybean oil | | | | | | |
| | (150) | | | | | | |
| | CH₃OK | | | | | | |
| 6-3 | Glycerol | 4 | 430 | 8.2 | 0.5 | 649 | 910 |
| | (100) | | | | | | |
| | Methyl | | | | | | |
| | ricinoleate | | | | | | |
| | (220) | | | | | | |
| | CH₃OK | | | | | | |
| 6-4 | Glycerol | 4 | 213 | 3.5 | 1 | 618 | 882 |
| | (100) | | | | | | |
| | Methyl | | | | | | |
| | soyate (250) | | | | | | |
| | CH₃OK | | | | | | |
| 6-5 | Glycerol | 6 | 260 | 7.8 | 0.9 | 625 | 940 |
| | (150) | | | | | | |
| | Sunflower | | | | | | |
| | oil (150) | | | | | | |
| | CH₃OK | | | | | | |
| 6-6 | Glycerol | 6 | 240 | 6.7 | 1.1 | 610 | 920 |
| | (150) | | | | | | |
| | Corn oil | | | | | | |
| | (150) | | | | | | |
| | CH₃OK | | | | | | |
| 6-7 | Glycerol | 6 | 238 | 4.2 | 1 | 590 | 914 |
| | (150) | | | | | | |
| | High oleic | | | | | | |
| | safflower oil | | | | | | |
| | (150) | | | | | | |
| | CH₃OK | | | | | | |
| 6-8 | Glycerol | 6 | 240 | 7.2 | 1.1 | 622 | 938 |
| | (150) | | | | | | |
| | Canola oil | | | | | | |
| | (150) | | | | | | |
| | CH₃OK | | | | | | |
| 6-9 | Glycerol | 6 | 440 | 12.4 | 1.2 | 655 | 940 |
| | (100) | | | | | | |
| | 9(10)- | | | | | | |
| | hydroxy- | | | | | | |
| | 10(9)- | | | | | | |
| | methoxy | | | | | | |
| | methyl | | | | | | |
| | stearate | | | | | | |
| | (200) | | | | | | |
| | CH₃OK | | | | | | |
| 6-10 | Glycerol | 6 | 420 | 16.8 | 0.9 | 660 | 955 |
| | (100) | | | | | | |
| | 9(10)- | | | | | | |
| | methylol- | | | | | | |
| | methyl | | | | | | |
| | stearate | | | | | | |
| | (200) | | | | | | |
| | CH₃OK | | | | | | |

Further embodiments of the invention are described by the untested examples listed in Table 6A. The following examples are provided for the purpose of illustration not limitation, and the changes reflected are equally applicable to each of the compositions described in Examples 6-1 through 6-10.

TABLE 6A
Untested Examples of Polyols by Polycondensation
of Glycerol With Vegetable Oil Derivatives

| | Vegetable Oil | Glycerol | | Time & |
|---|---|---|---|---|
| No. | (grams) | grams | Catalyst | Temperature |
| 6A-1 | Castor Oil | 180 | CH₃OK | 6 hours, 160° C. |
| | (120) | | | |
| 6A-2 | Castor Oil | 180 | CH₃OK | 4 hours, 190° C. |
| | (120) | | | |
| 6A-3 | Castor Oil | 15 | CH₃OK | 6 hours, 160° C. |
| | (285) | | | |
| 6A-4 | Castor Oil | 15 | CH₃OK | 4 hours, 190° C. |
| | (285) | | | |
| 6A-5 | Polyol-204 | 180 | KOH | 6 hours, 160° C. |
| | (120) | | | |
| 6A-6 | Polyol-204 | 180 | KOH | 4 hours, 190° C. |
| | (120) | | | |
| 6A-7 | Polyol-204 | 15 | KOH | 6 hours, 160° C. |
| | (285) | | | |
| 6A-8 | Polyol-204 | 15 | KOH | 4 hours, 190° C. |
| | (285) | | | |
| 6A-9 | Castor Oil | 180 | Ca(OH)₂ | 6 hours, 160° C. |
| | (120) | | | |
| 6A-10 | Castor Oil | 180 | Ca(OH)₂ | 4 hours, 190° C. |
| | (120) | | | |
| 6A-11 | Castor Oil | 15 | Ca(OH)₂ | 6 hours, 160° C. |
| | (285) | | | |
| 6A-12 | Castor Oil | 15 | Ca(OH)₂ | 4 hours, 190° C. |
| | (285) | | | |
| 6A-13 | Polyol-173 | 180 | CH₃ONa | 6 hours, 160° C. |
| | (120) | | | |
| 6A-14 | Polyol-173 | 180 | CH₃ONa | 4 hours, 190° C. |
| | (120) | | | |
| 6A-15 | Polyol-173 | 15 | CH₃ONa | 6 hours, 160° C. |
| | (285) | | | |
| 6A-16 | Polyol-173 | 15 | CH₃ONa | 4 hours, 190° C. |
| | (285) | | | |
| 6A-17 | Soybean Oil | 180 | NaOH | 6 hours, 160° C. |
| | (120) | | | |
| 6A-18 | Soybean Oil | 180 | NaOH | 4 hours, 190° C. |
| | (120) | | | |
| 6A-19 | Soybean Oil | 15 | NaOH | 6 hours, 160° C. |
| | (285) | | | |
| 6A-20 | Soybean Oil | 15 | NaOH | 4 hours, 190° C. |
| | (285) | | | |

In another embodiment, direct synthesis of polyglycerol esters is accomplished by reacting a desired amount of glycerol with a desired amount of benzoic acid or phthalic anhydride with or without an alkaline catalyst, such as potassium methoxide (CH₃OK), or an acid catalyst, such as p-toluene sulfonic acid (p-TsOH), at a temperature of approximately 80° C.-250° C., preferably approximately 200° C.-240° C., for about 1-9 hours, preferably about 2-6 hours. Examples are shown in Table 7.

TABLE 7
Polyols by Polycondensation of Glycerol With Aromatic Acids and Anhydrides

| | Reactants & | | | | | | | |
|---|---|---|---|---|---|---|---|---|
| | Catalyst | Temp. | | OH no. | Viscosity | Acid | OH/acid | Aromatic |
| No. | (grams) | & Time | | mg KOH/g | Pa · s., 25° C. | Value | ratio | content % |
| 7-1 | Glycerol | 240° | C. | 638 | 1.48 | 3.5 | 10:1 | 18 |
| | (50), | 9 | hrs. | | | | | |
| | Benzoic | | | | | | | |
| | acid (20), | | | | | | | |
| | CH₃OK | | | | | | | |
| | (0.7) | | | | | | | |
| 7-2 | Glycerol | 240° | C. | 549 | 1 | 2.6 | 10:1 | 18 |
| | (50), | 6 | hrs. | | | | | |
| | Benzoic | | | | | | | |
| | acid (20), | | | | | | | |
| | no catalyst, | | | | | | | |
| | Glycidol | | | | | | | |
| | (0)* | | | | | | | |
| 7-3 | Glycerol | 240° | C. | 516 | 1.2 | 2 | 9.75:1 | 21 |
| | (100), | 2.5 | hrs. | | | | | |
| | Benzoic | | | | | | | |
| | acid (50), | | | | | | | |
| | no catalyst, | | | | | | | |
| | Glycidol | | | | | | | |
| | (1.5)* | | | | | | | |
| 7-4 | Glycerol | 240° | C. | 534 | 2.2 | 0.9 | 6.6:1 | 24 |
| | (100), | 3 | hrs. | | | | | |
| | Benzoic | | | | | | | |
| | acid (60), | | | | | | | |
| | no catalyst, | | | | | | | |
| | Glycidol | | | | | | | |
| | (3.2)* | | | | | | | |
| 7-5 | Glycerol | 240° | C. | 452 | 1.5 | 1.4 | 4.9:1 | 28 |
| | (100), | 2.5 | hrs. | | | | | |
| | Benzoic | | | | | | | |
| | acid (80), | | | | | | | |
| | no catalyst, | | | | | | | |
| | Glycidol | | | | | | | |
| | (3.6)* | | | | | | | |
| 7-6 | Glycerol | 240° | C. | 381 | 1.5 | 0.8 | 3.94:1 | 32 |
| | (100), | 2.5 | hrs. | | | | | |
| | Benzoic | | | | | | | |
| | acid (100), | | | | | | | |
| | no catalyst, | | | | | | | |
| | Glycidol | | | | | | | |
| | (3.6)* | | | | | | | |
| 7-7 | Glycerol | 240° | C. | n/a | 682 | 11.7 | 7.2:1 | 21 |
| | (50), | 1 hr. | | | | | | |
| | Benzoic | | | | | | | |
| | acid (25), | | | | | | | |
| | p-TsOH | | | | | | | |
| | (0.5) | | | | | | | |
| 7-8 | Glycerol | 200° | C. | 364 | 35.9 | 11.8 | 7.2:1 | 21 |
| | (50), | 2 | hrs. | | | | | |
| | Benzoic | | | | | | | |
| | acid (25), | | | | | | | |
| | p-TsOH | | | | | | | |
| | (0.5) | | | | | | | |
| 7-9 | Glycerol | 160° | C. | 664 | 1 | 17.4 | 7.2:1 | 21 |
| | (50), | 3 | hrs. | | | | | |
| | Benzoic | | | | | | | |
| | acid (25), | | | | | | | |
| | p-TsOH | | | | | | | |
| | (0.5) | | | | | | | |
| 7-10 | Glycerol | 120° | C. | 863 | 0.4 | 80.2 | 7.2:1 | 21 |
| | (50), | 4 | hrs. | | | | | |
| | Benzoic | | | | | | | |
| | acid (25), | | | | | | | |
| | p-TsOH | | | | | | | |
| | (0.5) | | | | | | | |
| 7-11 | Glycerol | 80° | C. | 783 | 0.2 | 102 | 7.2:1 | 21 |
| | (50), | 5 | hrs. | | | | | |
| | Benzoic | | | | | | | |
| | acid (25), | | | | | | | |
| | p-TsOH | | | | | | | |
| | (0.5) | | | | | | | |
| 7-12 | Glycerol | 240° | C. | 771 | 1.4 | 4.6 | 7.2:1 | 21 |
| | (50), | 2 | hrs. | | | | | |
| | Benzoic | | | | | | | |
| | acid (25), | | | | | | | |
| | p-TsOH | | | | | | | |
| | (0.1) | | | | | | | |
| 7-13 | Glycerol | 240° | C. | 575 | 3.3 | 6.6 | 7.2:1 | 21 |
| | (50), | 2 | hrs. | | | | | |
| | Benzoic | | | | | | | |
| | acid (25), | | | | | | | |
| | p-TsOH | | | | | | | |
| | (0.2) | | | | | | | |
| 7-14 | Glycerol | 240° | C. | 572 | 63.4 | 6 | 4.9:1 | 17 |
| | (50), | 4 | hrs. | | | | | |
| | Phthalic | | | | | | | |
| | anhydride | | | | | | | |
| | (24.3), no | | | | | | | |
| | catalyst | | | | | | | |
| 7-15 | Glycerol | 240° | C. | 846 | 10 | 1.2 | 7.9:1 | 12 |
| | (100), | 2.5 | hrs. | | | | | |
| | Phthalic | | | | | | | |
| | anhydride | | | | | | | |
| | (30), no | | | | | | | |
| | catalyst | | | | | | | |
| 7-16 | Glycerol | 240° | C. | 430 | 13.2 | 1.2 | 4.9:1 | 21.3 |
| | (100), | 2.5 | hrs. | | | | | |
| | Phthalic | | | | | | | |
| | anhydride | | | | | | | |
| | (30), | | | | | | | |
| | Benzoic | | | | | | | |
| | acid (30), | | | | | | | |
| | no catalyst | | | | | | | |

*In these examples, glycidol was added at the end of the reaction to decrease acidity.

As shown in Table 7A below, time and temperature variables exhibit a significant effect on p-toluene sulfonic acid catalyzed direct polycondensation-esterification of glycerol with benzoic acid. Effective p-toluene sulfonic acid catalyst levels are in the range of about 0.1% by weight to about 0.7% by weight. At high catalyst concentrations and temperatures above about 200° C., significant amounts of higher oligomers of polyglycerol are produced resulting in polyols with relatively high viscosity. At high catalyst concentrations and temperatures below about 200° C., formation of higher oligomers was substantially decreased resulting in polyols with lower viscosity, but having relatively high levels of unreacted glycerol and unreacted benzoic acid. In an exemplary embodiment, reaction conditions are about 2 hours at about 240° C. and p-toluene sulfonic acid catalyst levels of about 0.13%-0.27% by weight, where the produced polyols have low viscosities, high hydroxyl numbers, and low levels of unreacted benzoic acid.

TABLE 7A
Characteristics of Polyols by Polycondensation of Glycerol With Benzoic Acid

| | | | | | | | | Viscosity |
|---|---|---|---|---|---|---|---|---|
| Example | Temp. & | | p- | | | Higher | Benzoic | Pa · s., |
| No. | Time | | TsOH % | Glycerol % | Dimer % | Oligomers % | Acid % | 25° C. |
| 7-11 | 80° | C. | 0.67 | 82 | 6 | 0 | 12 | 0.2 |
| | 5 | hrs. | | | | | | |
| 7-10 | 120° | C. | 0.67 | 29 | 38 | 3 | 30 | 0.4 |
| | 4 | hrs. | | | | | | |
| 7-9 | 160° | C. | 0.67 | 19 | 50 | 25 | 6 | 1 |
| | 3 | hrs. | | | | | | |
| 7-8 | 200° | C. | 0.67 | 2 | 20 | 73 | 4 | 35.9 |
| | 2 | hrs. | | | | | | |
| 7-7 | 240° | C. | 0.67 | 1 | 22 | 72 | 4 | 682 |
| | 1 | hr. | | | | | | |
| 7-12 | 240° | C. | 0.13 | 16 | 57 | 27 | 0 | 1.4 |
| | 2 | hrs. | | | | | | |
| 7-13 | 240° | C. | 0.27 | 12 | 43 | 43 | 2 | 3.3 |
| | 2 | hrs. | | | | | | |

The various embodiments of the polyglycerol based polyols of the invention are miscible with organic compounds and partially miscible with water, thus having the right hydrophilic/hydrophobic balance for use in polyurethanes; have good clarity and storage stability; have low color, preferably not greater than light yellow; have low viscosity, preferably below 10 Pa.s at 25° C.; hydroxyl functionality of approximately 2-10, preferably 3-6; have low acid value; preferably less than about 2 mg KOH/g; have glycerin content of approximately 50% or greater; have high bio-based renewable resource content, preferably approximately 80% by weight or greater; and have adequate reactivity with isocyanates for use in polyurethane foam formulations. In some embodiments, aromatic containing polyols are useful to increase polyurethane foam rigidity and improve fire resistance. In other embodiments the polyols are based on polyglycerol modified with glycols to increase spacing between OH groups that are useful in making polyurethanes having enhanced flexibility. The various embodiments of the polyglycerol based polyols of the invention having hydroxyl numbers above about 300 mg KOH/g, preferably about 300-600 mg KOH/g, are useful for making rigid polyurethane foams. Polyols having hydroxyl numbers below about 300 mg KOH/g are useful for making flexible polyurethanes.

The high functionality polyols obtained from the transesterification and co-polycondensation processes described above can be used for making rigid polyurethane foams. The polyol is mixed first with catalysts (for example: POLYCAT® 5, POLYCAT® 77, and DABCO® T-12), then with silicone surfactant (for example: DABCO® DC-198, SPI 200, or DC 5604), and water as chemical blowing agent. The resulting polyol mixture, is then mixed with a commercial grade of 4,4′-diphenylmethane diisocyanate (“MDI”; for example Bayer MONDUR® CD, or Huntsman RUBINATE® M), with stirring at approximately 15° C. to approximately 30° C., then transferred to a suitable container for curing at approximately 10° C. to approximately 110° C., preferably approximately 20° C. to approximately 30° C. The MDI is added in an amount of approximately 0.5-1.6 equivalents of NCO to 1 equivalent of total OH including water, preferably approximately 1-1.15 equivalents of NCO to 1 equivalent of total OH including water. The resulting rigid polyurethane foams are characterized by measuring density and compression strength. Examples are shown in Table 8, and foam properties are shown in Table 8A.

TABLE 8
Formulations for Rigid Polyurethane Foams

| | | DABCO | | DABCO | | | Total |
|---|---|---|---|---|---|---|---|
| | Polyol | DC-198 | POLYCAT 5 | T-12 | Water | MDI | NCO/OH |
| No. | (grams) | (grams) | (grams) | (grams) | (grams) | (grams) | Ratio |
| 8-1 | Example 3- | 1.5 | 0.5 | 0.5 | 3 | 143.5 | 1.1/1 |
| | 5 (100) | | | | | | |
| 8-2 | Example 6- | 1.5 | 0.5 | 0.5 | 3 | 125 | 1.1/1 |
| | 1 (100) | | | | | | |
| 8-3 | Example 3- | 1.5 | 0.5 | 0.5 | 3 | 288 | 0.95:1 |
| | 22 (100) | | | | | | |
| 8-4 | Example 3- | 1.5 | 0.1 | 0.5 | 3 | 166.5 | 0.55/1 |
| | 22 (100) | | | | | | |
| 8-5 | Example 6- | 1.5 | 0.1 | 0.1 | 3 | 109 | 1.15/1 |
| | 4 (100) | | | | | | |
| 8-6 | Example 6- | 1.5 | 0.1 | 0.1 | 3 | 113 | 1.1/1 |
| | 2 (100) | | | | | | |
| 8-7 | Example 5- | 0.5 | 0.2 | POLYCAT | 1 | 39 | 1.56/1 |
| | 1 (20) | | | 77 | | | |
| | | | | 0.2 | | | |
| 8-8 | Example 5- | 0.5 | 0.2 | POLYCAT | 1 | 40 | 1.28/1 |
| | 2 (20) | | | 77 | | | |
| | | | | 0.2 | | | |
| 8-9 | Example 5- | 0.5 | 0.2 | POLYCAT | 1 | 39 | 1.04/1 |
| | 6 (20) | | | 77 | | | |
| | | | | 0.2 | | | |
| 8-10 | Example 5- | 0.5 | 0.2 | POLYCAT | 1 | 40 | 1.29/1 |
| | 7 (20) | | | 77 | | | |
| | | | | 0.2 | | | |
| 8-11 | Example 5- | 0.5 | 0.2 | POLYCAT | 1 | 39 | 1.35/1 |
| | 8 (20) | | | 77 | | | |
| | | | | 0.2 | | | |
| 8-12 | Example 7- | SPI 200 | 0.2 | POLYCAT | 1 | 58 | 1.48/1 |
| | 3 (20) | 0.3 | | 77 | | | |
| | | | | 0.2 | | | |
| 8-13 | Example 7- | SPI 200 | 0.2 | POLYCAT | 1 | 44 | 1.1/1 |
| | 4 (20) | 0.3 | | 77 | | | |
| | | | | 0.2 | | | |
| 8-14 | Example 7- | SPI 200 | 0.2 | POLYCAT | 1 | 40 | 1.11/1 |
| | 5 (20) | 0.3 | | 77 | | | |
| | | | | 0.2 | | | |
| 8-15 | Example 7- | SPI 200 | 0.2 | POLYCAT | 1 | 37 | 1.13/1 |
| | 6 (20) | 0.3 | | 77 | | | |
| | | | | 0.2 | | | |
| 8-16 | Example 7- | SPI 200 | 0.2 | POLYCAT | 1 | 37 | 1.05/1 |
| | 16 (20) | 0.3 | | 77 | | | |
| | | | | 0.2 | | | |

TABLE 8A
Properties of Rigid Polyurethane Foams

| Example | | Density | Compression Strength |
|---|---|---|---|
| No. | Closed Cell % | Kg/m³ | kPa |
| 8-1 | | 51.2 | 366 |
| 8-2 | | 36.1 | 138 |
| 8-3 | | 72.3 | 421 |
| 8-4 | | 55.9 | 469 |
| 8-5 | | 44.6 | 210 |
| 8-6 | | 49.6 | 189 |
| 8-7 | 83 | 23 | 71 |
| 8-8 | 25 | 21 | 86 |
| 8-9 | 18 | 24.7 | 110 |
| 8-10 | 65 | 24 | 79 |
| 8-11 | 35 | 21.3 | 67 |
| 8-12 | 94 | 30.3 | 146 |
| 8-13 | 90 | 26.3 | 127 |
| 8-14 | 79 | 33.4 | 79 |
| 8-15 | 88 | 23.6 | 110 |
| 8-16 | 83 | 36.4 | 127 |

The high functionality polyols obtained from the transesterification and co-polycondensation processes described above can be used for making polyurethane cast resins. The desired amount of polyol is mixed first with a desired amount of one or more catalysts (POLYCAT 5, POLYCAT 77, and DABCO T-12). The resulting polyol mixture, is then mixed with a desired amount of a commercial grade of 4,4′-diphenylmethane diisocyanate (“MDI”; for example Bayer MONDUR CD, or Huntsman RUBINATE M), with stirring, at approximately 60° C. to approximately 65° C., then transferred to a suitable container for curing at approximately 100° C. to approximately 120° C., preferably approximately 110° C. to approximately 115° C. The resulting polyurethane cast resins are characterized by measuring mechanical properties, and swelling properties in toluene. Examples are shown in Table 9.

TABLE 9
Polyurethane Cast Resins

| | | | Mechanical properties | | | Swelling Properties | |
|---|---|---|---|---|---|---|---|
| | | | Break | | Tangent | in Toluene | |
| | | Tg, | Stress | Break | Modulus, | Average | Sol |
| No. | POLYOL | ° C. | Mpa | Elongation % | Mpa | Swelling Ratio | Fraction, % |
| 9-1 | Example 3-5 | 59 | 35 | 6.1 | 692 | 1.27 | — |
| 9-2 | Example 6-1 | 58 | 32 | 10.8 | 541 | 1.38 | 0.20 |
| 9-3 | Example 3-21 | 55 | 26 | 9.0 | 483 | 1.47 | 4.84 |
| 9-4 | Example 3-11 | 53 | 20 | 10.7 | 370 | 1.47 | 5.90 |
| 9-5 | Example 6-2 | 50 | 7.6 | 1.6 | 494 | 1.34 | 13.45 |

The invention provides high functionality polyols from substantially renewable resources, and polyurethane foams and polyurethane cast resins based on such polyols. From the above description of embodiments of the invention, those skilled in the art will perceive improvements, changes and modifications. Such improvements, changes and modifications within the skill of the art are intended to be covered by the appended claims. The invention is capable of embodiments in addition to those described and of being practiced and carried out in various ways. Also, it is to be understood that the phraseology and terminology employed herein, as well as the abstract, are for the purpose of description and should not be regarded as limiting.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention. Though some features of the invention may be claimed in dependency, each feature has merit when used independently.

We claim:

1. Polyols from renewable resources comprising carboxylic acid esters of polyglycerol wherein the polyols have hydroxyl numbers in the range of approximately 60 to approximately 1100 mg KOH/gram.

2. The polyols according to claim 1 wherein the polyglycerol includes from 2 to approximately 20 glyceryl repeating units.

3. The polyols according to claim 2 wherein at least one carboxylic acid is derived from the group consisting of C-1 to C-22 aliphatic and aromatic carboxylic acids, C-1 to C-22 aliphatic and aromatic carboxylic acid anhydrides, and alkyl esters of C-1 to C-22 aliphatic and aromatic carboxylic acids.

4. The polyols according to claim 1 wherein at least one carboxylic acid is a fatty acid derived from natural oils including vegetable oils, animal fats and oils, fish oils, algae oils, and chemically modified vegetable oils containing alkyl chain hydroxyl groups; and the polyols have hydroxyl numbers in the range of approximately 300 to approximately 600 mg KOH/gram.

5. The polyols according to claim 1 wherein the carboxylic acid has at least one hydroxyl group on the alkyl chain.

6. The polyols according to claim 5 wherein the carboxylic acid is ricinoleic acid.

7. The polyols according to claim 5 wherein the carboxylic acid is 9(10)-hydroxy-10(9)-methoxy-stearic acid.

8. The polyols according to claim 1 wherein the polyols are derived from at least 80% by weight renewable resources; have at least 2 to approximately 10 hydroxyl groups per molecule; have viscosity less than approximately 30 Pa.s at 25° C.; have acid numbers below approximately 2 mg KOH/g; and are soluble in organic solvents.

9. The polyols according to claim 1 further comprising an additive to substantially eliminate turbidity and reduce viscosity to less than approximately 10 Pa.s at 25° C., wherein the additive is selected from the group consisting of propylene carbonate and dimethyl methylphosphonate.

10. The polyols according to claim 1 wherein the polyglycerol is a modified polyglycerol derived from co-polycondensation of glycerol with approximately 10% to 40% by weight of another polyhydroxy compound having 2 or more primary hydroxyl groups.

11. A process for making high functionality polyols from renewable resources comprising combining 5% to 60% by weight polyglycerol, 40% to 95% by weight of at least one carboxylic acid derivative, a desired amount of a catalyst, and heating for 2-10 hours at approximately 160° C. to approximately 250° C. to produce polyols having hydroxyl numbers in the range of approximately 300 to approximately 600 mg KOH/gram.

12. The process according to claim 11 comprising heating for approximately 4-6 hours at approximately 170° C. to approximately 230° C.

13. The process according to claim 11 wherein at least one carboxylic acid derivative is a natural oil derivative selected from the group consisting of canola oil, castor oil, coconut oil, corn oil, cottonseed oil, linseed oil, olive oil, palm oil, peanut oil, rapeseed oil, safflower oil, sesame oil, soybean oil, sunflower oil; animal fats and oils, fish oils, algae oils; chemically modified vegetable oils, including vegetable oil polyols; and alkyl esters of fatty acids derived from natural oils.

14. The process according to claim 13 wherein two or more natural oil derivatives are combined with the polyglycerol.

15. The process according to claim 13 wherein the catalyst is a tin catalyst.

16. The process according to claim 13 wherein the catalyst is an alkali-metal alkoxide.

17. The process according to claim 11 wherein the carboxylic acid derivative is one or more components selected from the group consisting of C-1 to C-22 aliphatic and aromatic carboxylic acids, C-1 to C-22 aliphatic and aromatic carboxylic acid anhydrides, and alkyl esters of C-1 to C-22 aliphatic and aromatic carboxylic acids.

18. The process according to claim 17 further including at least one natural oil derivative.

19. The process according to claim 17 wherein at least one catalyst is derived from the group consisting of acid catalysts, alkaline catalysts, tin catalysts and titanium catalysts.

20. The process according to claim 17 wherein no catalyst is added.

21. The process according to claim 11 wherein the polyglycerol includes from 2 to approximately 20 repeating units.

22. The process according to claim 11 wherein the polyols have viscosity less than approximately 30 Pa.s at 25° C.; have acid numbers below approximately 2 mg KOH/g; and are soluble in organic solvents.

23. A process for making high functionality polyols from renewable resources comprising combining 5% to 60% by weight glycerol; 40% to 95% by weight of at least one carboxylic acid derivative; at least one catalyst; and heating for a desired amount of time at approximately 160° C. to approximately 270° C. to produce polyols having hydroxyl numbers in the range of approximately 300 to approximately 600 mg KOH/gram.

24. The process according to claim 23 wherein at least one carboxylic acid derivative is a natural oil derivative selected from the group consisting of canola oil, castor oil, coconut oil, corn oil, cottonseed oil, linseed oil, olive oil, palm oil, peanut oil, rapeseed oil, safflower oil, sesame oil, soybean oil, sunflower oil; animal fats and oils, fish oils, algae oils; chemically modified vegetable oils, including vegetable oil polyols; and lower alkyl esters of fatty acids derived from natural oils.

25. The process according to claim 23 wherein the catalyst is an alkali-metal alkoxide.

26. The process according to claim 23 wherein the carboxylic acid derivative is one or more components selected from the group consisting of C-1 to C-22 aliphatic and aromatic carboxylic acids, C-1 to C-22 aliphatic and aromatic carboxylic acid anhydrides, and lower alkyl esters of C-1 to C-22 aliphatic and aromatic carboxylic acids.

27. The process according to claim 26 further including at least one natural oil derivative.

28. The process according to claim 26 wherein at least one catalyst is derived from the group consisting of acid catalysts, alkaline catalysts, tin catalysts and titanium catalysts.

29. The process according to claim 26 wherein the catalyst is p-toluene sulfonic acid.

30. The process according to claim 23 wherein the polyols have viscosity less than approximately 30 Pa.s at 25° C.; have acid numbers below approximately 2 mg KOH/g; and are soluble in organic solvents.

31. A process for making polyglycerol comprising combining a desired amount of glycerol obtained as a by-product from bio-diesel production with a desired amount of catalyst; heating the combined glycerol and catalyst to approximately 240° C. to 270° C.; and collecting a desired amount of water of condensation to provide polyglycerol having from 2 to approximately 20 glyceryl repeating units.

32. The process according to claim 31 wherein the catalyst is an alkaline catalyst.

33. The process according to claim 32 wherein the glycerol and catalyst are heated at approximately 250° C. for up to approximately 10 hours.

34. The process according to claim 32 wherein hyper-branched polyglycerols are prepared by condensing approximately 60% to 90% by weight glycerol with approximately 10% to 40% by weight of a polyhydroxy compound.

35. The process according to claim 34 wherein the polyhydroxy compound is trimethylol propane.

36. The process according to claim 34 wherein the polyhydroxy compound is pentaerythritol.

37. The process according to claim 31 wherein polyglycerols are prepared by condensing approximately 60% to 90% by weight glycerol with approximately 10% to 40% by weight of a glycol.

38. The process according to claim 37 wherein the glycol is diethylene glycol and the catalyst is p-toluene sulfonic acid.

39. Polyurethane compositions from renewable resources comprising urethane polymers of polyols derived from carboxylic acid esters of polyglycerol.

40. The composition according to claim 39 comprising rigid polyurethane foam compositions wherein the polyol is a copolymer of glycerol and a vegetable oil, and the polyol having hydroxyl number in the range of approximately 300 to approximately 600 mg KOH/gram.

41. The composition according to claim 39 comprising rigid polyurethane foam compositions wherein the polyols are derived from transesterification of a vegetable oil with polyglycerol, and the polyol having hydroxyl number in the range of approximately 300 to approximately 600 mg KOH/gram.

42. A process of making polyurethane compositions from renewable resources comprising reaction of an aromatic poly-isocyanate with a polyol wherein the polyol is derived from the group consisting of transesterification of polyglycerol with natural oil derivatives; esterification of polyglycerol with C-1 to C-22 aliphatic and aromatic carboxylic acids, C-1 to C-22 aliphatic and aromatic carboxylic acid anhydrides, and lower alkyl esters of C-1 to C-22 aliphatic and aromatic carboxylic acids; polycondensation of glycerol with natural oil derivatives; and polycondensation of glycerol with C-1 to C-22 aliphatic and aromatic carboxylic acids, C-1 to C-22 aliphatic and aromatic carboxylic acid anhydrides, and lower alkyl esters of C-1 to C-22 aliphatic and aromatic carboxylic acids.
